(19) United States
(12) Patent Application Publication
(10) Pub. No.: US 2024/0366496 A1
(43) Pub. Date: Nov. 7, 2024

(54) COSMETIC COMPOSITION COMPRISING AT LEAST ONE BIRCH EXTRACT AND AT LEAST ONE BEECH BUD EXTRACT

(71) Applicant: CREDO, BORDEAUX (FR)

(72) Inventor: Anne-Sophie NARDY, BORDEAUX (FR)

(21) Appl. No.: 18/552,852

(22) PCT Filed: Mar. 25, 2022
(86) PCT No.: PCT/FR2022/050559

§ 371 Date: Sep. 27, 2023

(30) Foreign Application Priority Data
Apr. 2, 2021 (FR) 2103424

(51) Int. Cl. A61K 8/9789; A61Q 19/08

(52) U.S. Cl. CPC A61K 8/9789; A61Q 19/08; A61K 2800/74; A61K 2800/805

(57) ABSTRACT

The invention relates to a cosmetic composition comprising at least one birch sap extract, and at least one extract chosen from an aqueous extract of beech buds and an oily extract of beech buds, and at least one cosmetically acceptable vehicle.

### TECHNICAL AREA

The invention relates to a cosmetic composition comprising at least one birch sap extract, preferably glycerinated, and at least one extract chosen from an aqueous extract of beech buds and an oily extract of beech buds, and at least one cosmetically acceptable vehicle.

The present invention also relates to a non-therapeutic cosmetic care method comprising the application to the skin or mucous membranes of a cosmetic composition comprising at least one aqueous extract of beech bud, one oily extract of beech bud and/or at least one extract of birch.

The present invention finds application in the field of cosmetics and dermatology, more particularly in cutaneous cosmetics.

In the description below, references in square brackets ([ ]) refer to the list of references at the end of the text.

### STATE OF THE ART

The skin is an organ in its own right, playing an essential role not only in protecting the body against external aggression, but also in its aesthetic and emotional appeal. The skin is an organ of exchange. Both its visual and tactile aspects play an important role.

The skin as an organ is directly exposed to external conditions, including temperature, light, humidity, UV rays and pathogens. Under normal, healthy conditions, many attributes of human skin follow a periodicity: hydration and trans-epidermal water loss, capillary blood flow, sebum production, temperature, surface pH and keratinocyte proliferation rate.

With age, keratinocyte renewal slows considerably, and the protein composition of the horny envelope responsible for barrier function is profoundly altered. It is therefore important to restore keratinocyte proliferation and differentiation potential in order to restore epidermal homeostasis and the skin's barrier function, which are disrupted by skin aging.

It's also a well-known fact that as we age, our skin loses its firmness and elasticity, which can lead to the appearance of wrinkles, both on the face and all over the body.

The state of the art includes numerous cosmetic compositions for the treatment of skin ageing and/or skin firming. However, these compositions have relative efficacy, and in some cases have even been challenged by consumer associations. The relative efficacy of known compositions can lead to a need for individuals to resort to surgical procedures such as hyaluronic acid and/or botulinum toxin injections to mask wrinkles.

### DESCRIPTION OF THE INVENTION

The present invention is precisely designed to meet these needs and drawbacks of the prior art.

The inventors of the present invention are indeed the very first to have highlighted, in a completely unexpected way, that a cosmetic composition according to the invention advantageously enables an action on the aesthetics and ageing of the skin and/or its appendages and/or mucous membranes, in particular to provide radiance, suppleness, finesse, smoothness and/or softness. This action can be achieved in particular by stimulating the proliferation/regeneration of epidermal cells and hydration, while protecting against external aggressions (e.g. pollution) or oxidative stress, by reinforcing the skin's barrier function.

The object of the present invention is therefore a cosmetic composition comprising at least one extract of birch sap, and at least one extract chosen from an aqueous extract of beech bud and an oily extract of beech bud, and a cosmetically acceptable carrier.

According to the present invention, “birch” refers to a species of deciduous tree in the Betulaceae family and the Betula genus. In particular, in Europe, this could be Betula alba, Betula pendula or Betula pubescens. The sap, leaves and bark have diuretic properties and are generally used to treat skin disorders.

According to the present invention, “beech” or “common beech” (Fagus sylvatica) is a species of deciduous tree native to Europe, belonging to the Fagaceae family. Its astringent bark is used as a febrifuge. In powder form, the bark is used to treat gout, rheumatism, dropsy and intractable skin conditions. Known in particular for its targeted action on the kidneys and adipose tissue, the bud helps to “cleanse” the body, resulting in a draining and energizing effect. In cosmetics, bud extracts are used to energize the skin and improve its radiance, boost cell oxygenation and stimulate metabolism.

According to the present invention, “extract” means any form of extract known to the person skilled in the art. It may be, for example, an oily extract, an aqueous extract, a hydroalcoholic extract, a hydro-glycerin extract, a glycerin extract, a supercritical extract, an essential oil or any mixture thereof. Such extracts can be obtained from any part of a plant, for example flowers or flowering tops, leaves, seeds, roots, stems, pericarp, buds or sap.

According to the present invention, an “aqueous extract”, in particular an “aqueous extract of beech buds”, can be obtained by any process known to the person skilled in the art. It may, for example, be an aqueous extract obtained by aqueous extraction, by hydro-distillation, by hydro-glycerin, hydro-glycolic or hydro-alcoholic extraction, supercritical water extraction. For example, it may be an aqueous extract obtained using the process described in the book “Eco-extraction du végétal” [1]. It may also be an aqueous extract of beech buds obtained by a supercritical water extraction process. It may also be a commercially available aqueous extract of beech buds.

According to the present invention, an “oil extract”, in particular a “beech bud oil extract”, can be obtained by any suitable process known to the skilled person. It may, for example, be an oily extract obtained by microwave oil extraction, hyper-frequency oil extraction, supercritical oil extraction, maceration in an oily solvent or ultrasound-assisted oil extraction. For example, it may be an oily extract obtained by maceration in a vegetable oil, for example in deodorized sunflower oil, followed by filtration, for example through a filter comprising pores with a diameter of 0.2 μm. This may be a commercially available oily extract of beech buds.

According to a particular embodiment of the present invention, the cosmetic composition according to the invention comprises at least an extract of birch sap and an aqueous extract of beech bud, and optionally an oily extract of beech bud.

According to another particular embodiment of the present invention, the cosmetic composition according to the invention comprises at least one birch sap extract, one aqueous beech bud extract, one oily beech bud extract, and at least one cosmetically acceptable vehicle.

According to a particular embodiment of the present invention, said birch sap extract is a glycerin extract of birch sap.

According to the present invention, a “birch sap glycerin extract” can be obtained by any suitable process known to the skilled person. It may, for example, be a glycerin extract obtained by a process comprising maceration of a natural plant extract (e.g. sap) in a mixture of water, alcohol and glycerin. It may also be a mixture of a natural plant extract (e.g. sap) with glycerin.

According to a particular embodiment of the present invention, the cosmetic composition according to the invention comprises from 0.2 to 5%, preferably from 0.5 to 2%, preferably 1% by weight of said aqueous extract of beech bud relative to the total weight of the composition.

According to a particular embodiment of the present invention, the cosmetic composition according to the invention comprises from 0.2 to 5%, preferably from 0.5 to 2%, preferably 1%, by weight of said beech bud oil extract relative to the total weight of the composition.

According to a particular embodiment of the present invention, the cosmetic composition according to the invention comprises from 0.5 to 6%, preferably from 1 to 3%, preferably 2%, by weight of said birch sap glycerol extract relative to the total weight of the composition.

The cosmetic composition according to the invention can be in any form suitable for cosmetic application. Advantageously, the composition is a composition for topical use. For example, it may be a composition in a form chosen from an ointment, cream, oil, milk, ointment, powder, soaked pad, solution, gel, serum, balm, butter, lotion, suspension, soap and emulsion (oil-in-water, or water-in-oil, or mixtures thereof). These formulations, which can be used to implement the present invention, are known to formulators in the state of the art.

The cosmetic composition according to the invention may comprise one or more adjuvants known to the person skilled in the art. These may include, for example, one or more adjuvant(s) chosen from ester-type agents, moisturizing agents, water, humectants, conditioning agents, emollient agents such as vegetable oils, mineral thickening agents, organic thickening agents, whether associative or not, perfumes, preservatives, ceramides and pseudo-ceramides, vitamins and provitamins, amino acids, proteins, plant extracts, sequestering agents, alkalizing agents, acidifying agents, reducing agents, oxidizing agents, mineral fillers, colorants or any other adjuvant listed in the INCI dictionary (International Nomenclature of Cosmetic Ingredients) published by the PCPC (Personal Care Products council).

Another object of the present invention is a device in a form chosen from a jar, a pump bottle, a wipe, a mask, a transdermal device, a patch, a spray, a capsule or a softgel, said device comprising a cosmetic composition according to the invention.

Another object of the present invention is a non-therapeutic cosmetic care method for improving the appearance of the skin and combating cutaneous ageing, comprising the application to the skin and/or its appendages and/or mucous membranes of a cosmetic composition according to the invention. Advantageously, said application improves hydration, nutrition, oxygenation and stimulation of the protective barrier function of the skin and/or its appendages and/or mucous membranes.

### EXAMPLES

### Example 1: Effects of the Compounds Beech Bud Oil Macerate, Beech Bud Aqueous Extract, White Birch Sap and their Combinations on Gene Expression in Normal Human Epidermal Keratinocytes

In this study, the effects of the compounds beech bud oil macerate, beech bud aqueous extract, white birch sap and the combinations “beech bud oil macerate+white birch sap” and “beech bud aqueous extract+white birch sap” were investigated in normal human epidermal keratinocytes (NHEK).

The effects of the compounds were assessed by RT-qPCR on the expression (mRNA) of 8 genes (including 2 reference genes) selected for their importance in keratinocyte physiology.

### Materials and Methods

### Biological Models

Normal human epidermal keratinocytes (NHEK)
Cell type: NHEK, reference Bioalternatives: K341 used at 3^{rd} passage
Culture conditions: 37° C., 5% CO₂
Culture medium: Serum-free medium (Keratinocyte-SFM) supplemented with epidermal growth factor (EGF) 0.25 ng/ml, pituitary extract (PE) 25 μg/ml, gentamycin 25 μg/ml.
Test medium: Serum-free medium (Keratinocyte-SFM) supplemented with gentamycin 25 μg/ml.

### Compounds Tested and Combinations

TABLE 1

| | | Stock or | NHEK |
|---|---|---|---|
| | Aspect/ | intermediate | concentration |
| Compound tested | Storage | solution | tested |
| Beech bud oily macerate | liquid | 10% in | 0.1% |
| Lot n°AF170720-M | storage: | THF | (Tetrahydrofuran |
| MV200714-1 | 4° C. | | (THF) − 1%) |
| Beech bud aqueous extract | liquid | 10% in | 0.3% |
| Batch no. A280520-F | storage: | test medium | |
| MV200714-2 | 4° C. | | |
| White birch sap | liquid | 10% in | 1% |
| Lot n°A240720-F | storage: | test medium | |
| MV200714-3 | 4° C. | | |

TABLE 2

| | Stock or intermediate | NHEK concentration |
|---|---|---|
| Association | solution | tested |
| Beech bud oily | see table above | 0.1% (THF − 1%) + |
| macerate + | | 1% (THF − 1%) |
| White birch sap | | |
| Beech bud aqueous | | 0.3% + 1% |
| extract + | | |
| White birch sap | | |

### Preliminary Cytotoxicity Tests

Cell type: NHEK in test medium
Incubation time: 24 hours
Evaluation parameter: reduction of 2-(2-methoxy-4-nitrophenyl)-3-(4-nitrophenyl)-5-(2,4-disulfophenyl)-2H-tetrazolium monosodium salt (WST-8) and morphological microscopic observations

At the end of the treatment, the cells were incubated in the presence of WST-8 (a highly water-soluble tetrazolium salt), reduced to an orange-colored, water-soluble product (formazan) by succinate dehydrogenase (a mitochondrial enzyme). This transformation is proportional to the number of living cells and their metabolic activity. Optical density (OD) was measured with a spectrophotometer at 450 nm (VERSAmax, Molecular Devices).

### Cultures and Treatments

Keratinocytes were seeded in 24-well plates and cultured in culture medium for 24 hours, then in test medium for a further 24 hours. After incubation, the culture medium was replaced by test medium containing or not (control) the compounds, associations or solvent control (1% THF) and the cells were incubated for a further 24 hours.

All conditions were performed in n=3.

At the end of incubation, culture supernatants were removed and cell mats rinsed with phosphate-buffered saline (PBS). Plates were immediately dry-frozen at −80° C.

### Differential Expression Analysis—PCR Matrix

Marker expression was assessed by RT-qPCR on total RNA extracted from the cell mats of each experimental condition (replicates of the same experimental condition were pooled before RNA extraction).

Transcript analysis was carried out in n=2 using research-dedicated “PCR arrays” adapted to the “screening” format (quantitative PCR array markers (mQPA) produced by Bioalternatives) and targeting 8 genes selected for their importance in keratinocyte physiology (“MV200714—mQPA NHEK-8 custom genes”).

### RNA Extraction and Reverse Transcription

Total RNA from each sample was extracted using TriPure Isolation Reagent® according to the protocol recommended by the supplier. RNA quantity and quality were assessed by capillary electrophoresis (Bioanalyzer 2100, Agilent).

Complementary DNA (cDNA) was synthesized by reverse transcription of total RNA in the presence of oligo (dT) and the reverse transcriptase enzyme Transcriptor Reverse Transcriptase (Roche). The quantities of cDNA were then adjusted before the PCR step.

### Quantitative PCR

PCR (polymerase chain reaction) reactions were performed by quantitative PCR (Light Cycler; Roche Molecular systems Inc.) and according to the procedures recommended by the supplier.

The reaction mixture (10 ml final) for each sample contained:
2.5 μl cDNA,
primers for the various markers used,
reaction mixture containing Taq polymerase enzyme, SYBR Green I label and MgCl₂.

### Data Processing

Raw data were transferred and processed in Microsoft Excel®.

### Preliminary Cytotoxicity

Formulas used in this report:

Standard error of the mean: esm=standard deviation (Sd)/√n

The standard error of the mean (esm) represents the standard deviation of the sample mean from the mean of the true population. The esm is calculated by dividing the Sd by the square root of the sample size.

$$Percentage of viability : viability ( \% ) = ( {OD}_{compound} / {OD}_{control} ) \times 100$$

### Quantitative PCR

Fluorescence incorporation into amplified DNA is measured continuously during PCR cycles. These measurements enable us to obtain fluorescence intensity curves as a function of PCR cycles, and thus to evaluate a relative expression value for each marker.

The number of cycles is determined from the “exit” points of the fluorescence curves. For the same marker analyzed, the later a sample exits (higher cycle number), the lower the initial mRNA copy number.

The relative expression value is determined according to the formula:

(½^{number of cycles})×10⁶.

The “MV200714—mQPA NHEK-8 custom gene” PCR templates contain 2 reference genes (housekeeping (HK) genes) used for data normalization: RPS28 (ribosomal protein S28) and GAPDH (glyceraldehyde-3-phosphate dehydrogenase). The reference genes selected are constitutively expressed genes whose expression levels are little or not at all affected by the treatments. Normalization of the expression level of the target markers is achieved by comparing their expression level with the average expression of these 2 reference genes.

### Results

### Preliminary Cytotoxicity Tests

The results of viability tests with WST-8 and the observation of cell mats led to the selection of the concentrations to be tested in the rest of the study (see Tables 3 to 5: effects of the compounds beech bud oily macerate, beech bud aqueous extract and white birch sap on keratinocyte viability after 24 hours).

TABLE 3

| | | | Beech buds oily macerate (Unit: %) | | | | | | | |
|---|---|---|---|---|---|---|---|---|---|---|
| | | | Stock solution prepared in 10% THF | | | | | | | |
| | Control | | 4.57−05^{E} | 1.37−04^{E} | 4.12−04^{E} | 0.0012 | 0.0037 | 0.011 | 0.033 | 0.1 |
| Viability (%) | 92 | 91 | 90 | 91 | 92 | 91 | 88 | 88 | 87 | 89 |
| | 105 | 102 | 103 | 97 | 103 | 97 | 103 | 99 | 106 | 109 |
| | 108 | 102 | 103 | 106 | 106 | 99 | 108 | 97 | 108 | 114 |

| | | | | | | | | | |
|---|---|---|---|---|---|---|---|---|---|
| Average (%) | 100 | 99 | 98 | 100 | 96 | 100 | 95 | 100 | 104 |
| esm (%) | 3 | 4 | 4 | 4 | 3 | 6 | 3 | 7 | 7 |
| Morphological | + | + | + | + | + | + | + | + | +, * |
| observation | | | | | | | | | |

TABLE 4

| | | | Aqueous extract of beech buds (Unit: %) | | | | | | | |
|---|---|---|---|---|---|---|---|---|---|---|
| | Control | | 0.0046 | 0.014 | 0.041 | 0.123 | 0.37 | 1.1 | 3.3 | 10 |
| Viability (%) | 98 | 99 | 97 | 94 | 93 | 89 | 79 | 77 | 68 | 21 |
| | 97 | 103 | 102 | 103 | 102 | 96 | 94 | 81 | 61 | 19 |
| | 105 | 97 | 102 | 104 | 102 | 95 | 90 | 83 | 62 | 19 |

| | | | | | | | | | |
|---|---|---|---|---|---|---|---|---|---|
| Average (%) | 100 | 100 | 100 | 99 | 93 | 88 | 80 | 64 | 20 |
| esm (%) | 1 | 2 | 3 | 3 | 2 | 5 | 2 | 2 | 1 |
| Morphological | + | + | + | + | + | + | + | +/−, * | +/−, * |
| observation | | | | | | | | | |

TABLE 5

| | | | White birch sap (Unit: %) | | | | | | | |
|---|---|---|---|---|---|---|---|---|---|---|
| | Control | | 0.0046 | 0.014 | 0.041 | 0.123 | 0.37 | 1.1 | 3.3 | 10 |
| Viability (%) | 96 | 108 | 105 | 100 | 103 | 99 | 97 | 89 | 68 | 43 |
| | 95 | 100 | 97 | 96 | 97 | 87 | 95 | 87 | 74 | 33 |
| | 98 | 103 | 104 | 99 | 101 | 107 | 111 | 90 | 66 | 33 |

| | | | | | | | | | |
|---|---|---|---|---|---|---|---|---|---|
| Average (%) | 100 | 102 | 99 | 101 | 98 | 101 | 89 | 69 | 36 |
| esm (%) | 2 | 2 | 1 | 2 | 6 | 5 | 1 | 2 | 3 |
| Morphological | + | + | + | + | + | + | + | +/−, * | +/−, * |
| observation | | | | | | | | | |

+: normal population;
+/−: growth reduction;
−: toxicity;
0: cell death
* morphological changes

### Effects on Gene Expression

The beech bud oil macerate compound was solubilized in THF, and the final concentration of the compound tested in the study contained 1% THF. A 1% THF solvent control was therefore performed on NHEK. THF tested at 1% on keratinocytes did not induce any significant change in the expression of the genes studied (see Table 6 below): Effects of solvent control (THF) on the expression of genes involved in normal human epidermal keratinocytes).

TABLE 6

| | | | 1% THF | |
|---|---|---|---|---|
| | | Control | | % control |
| STUDY | Genes | Cycles | Cycles | HK average |
| Household genes | RPS28 | 18.69 | 18.64 | 101 |
| | | 18.73 | 18.56 | |
| | GAPDH | 16.90 | 16.83 | 100 |
| | | 16.88 | 16.76 | |
| Marker of cell | KRT19 | 27.55 | 27.88 | 79 |
| regeneration/differentiation | | 27.80 | 27.93 | |
| Desquamation | KLK7 | 21.01 | 21.10 | 89 |
| | | 21.01 | 21.06 | |
| Hydration | FLG | 26.78 | 26.46 | 115 |
| | | 26.76 | 26.48 | |
| | AQP3 | 22.37 | 22.57 | 78 |
| | | 22.22 | 22.52 | |
| Innate immunity | S100A7 | 27.71 | 27.26 | 117 |
| | | 27.78 | 27.60 | |
| Response to oxidative | HMOX1 | 27.22 | 26.99 | 104 |
| and cellular stress | | 27.07 | 26.38 | |
| Barrier function | TGM1 | 23.09 | 23.42 | 77 |
| | | 23.12 | 23.35 | |

HK: reference
RPS28: ribosomal protein S28;
GAPDH: glyceraldehyde-3-phosphate dehydrogenase;
KRT19: keratin 19, type I;
KLK7: kallikrein-related peptidase 7;
FLG: profilaggrin;
AQP3: aquaporin 3 (Gill blood group);
S100A7: psoriasin or A7 calcium-binding protein S100;
HMOX1: heme oxygenase 1;
TGM1: transglutaminase 1

### Normal Human Epidermal Keratinocytes

The results are presented in Tables 7 to 9 (Effects of the compounds beech bud oily macerate, beech bud aqueous extract, white birch sap and the combinations “beech bud oily macerate+white birch sap” and “beech bud aqueous extract+white birch sap”, on gene expression in normal human dermal keratinocytes) below.

TABLE 7

| | | | Beech bud oily | | Beech bud aqueous | |
|---|---|---|---|---|---|---|
| | | | macerate | | extract | |
| | | | 0.1% | | 0.3% | |
| | | Control | | % control | | % control |
| STUDY | Genes | cycles | cycles | HK average | cycles | HK average |
| Household genes | RPS28 | 18.69 | 18.71 | 101 | 18.85 | 101 |
| | | 18.73 | 18.69 | | 18.84 | |
| | GAPDH | 16.90 | 16.94 | 100 | 17.07 | 100 |
| | | 16.88 | 16.85 | | 17.02 | |
| Marker of cell | KRT19 | 27.55 | 27.85 | 87 | 27.73 | 104 |
| regeneration/ | | 27.80 | 27.91 | | 27.81 | |
| differentiation | | | | | | |
| Desquamation | KLK7 | 21.01 | 21.49 | 71 | 20.73 | 136 |
| | | 21.01 | 21.53 | | 20.71 | |
| Hydration | FLG | 26.78 | 26.85 | 94 | 26.60 | 122 |
| | | 26.76 | 26.86 | | 26.68 | |
| | AQP3 | 22.37 | 22.56 | 80 | 22.69 | 85 |
| | | 22.22 | 22.67 | | 22.67 | |
| Innate immunity | S100A7 | 27.71 | 28.01 | 78 | 29.10 | 45 |
| | | 27.78 | 28.22 | | 29.00 | |
| Response to oxidative | HMOX1 | 27.22 | 26.91 | 121 | 27.04 | 120 |
| and cellular stress | | 27.07 | 26.84 | | 27.01 | |
| Barrier function | TGM1 | 23.09 | 23.75 | 64 | 23.22 | 103 |
| | | 23.12 | 23.74 | | 23.21 | |

TABLE 8

| | | | | Beech bud oily | | Beech bud aqueous | |
|---|---|---|---|---|---|---|---|
| | | | | macerate + White | | extract + White | |
| | | White birch sap | | birch sap | | birch sap | |
| | | 1% | | 0.1% + 1% | | 0.3% + 1% | |
| | | | % control | | % control | | % control |
| | | | HK | | HK | | HK |
| STUDY | Genes | cycles | average | cycles | average | cycles | average |
| Household genes | RPS28 | 18.79 | 97 | 18.77 | 94 | 19.29 | 105 |
| | | 18.74 | | 18.68 | | 19.18 | |
| | GAPDH | 16.99 | 101 | 16.81 | 102 | 17.94 | 99 |
| | | 16.80 | | 16.77 | | 17.53 | |
| Marker of cell | KRT19 | 26.95 | 172 | 25.77 | 354 | 26.59 | 301 |
| regeneration/ | | 26.86 | | 25.77 | | 26.77 | |
| differentiation | | | | | | | |
| Desquamation | KLK7 | 21.02 | 103 | 20.93 | 99 | 20.54 | 212 |
| | | 20.93 | | 20.96 | | 20.51 | |
| Hydration | FLG | 26.50 | 116 | 25.64 | 213 | 25.59 | 311 |
| | | 26.64 | | 25.57 | | 25.89 | |
| | AQP3 | 22.45 | 90 | 22.75 | 72 | 22.81 | 104 |
| | | 22.46 | | 22.65 | | 22.85 | |
| Innate immunity | S100A7 | 27.89 | 87 | 27.82 | 89 | 28.70 | 77 |
| | | 28.02 | | 27.85 | | 28.74 | |
| Response to | HMOX1 | 26.57 | 143 | 25.54 | 281 | 26.17 | 273 |
| oxidative and | | 26.71 | | 25.61 | | 26.43 | |
| cellular stress | | | | | | | |
| Barrier function | TGM1 | 23.00 | 109 | 22.96 | 105 | 22.51 | 229 |
| | | 23.00 | | 22.96 | | 22.51 | |

TABLE 9

| | | | | | Beech bud oily | |
|---|---|---|---|---|---|---|
| | | | Beech bud oily | | macerate + White | |
| | | Solvent | macerate | | birch sap | |
| | | control | 0.1% | | 0.3% + 1% | |
| | | (THF) | (THF 1%) | | (THF 1%) | |
| | | 1% | | % control | | % control |
| STUDY | Genes | cycles | cycles | HK average | cycles | HK average |
| Household genes | RPS28 | 18.64 | 18.71 | 100 | 18.77 | 93 |
| | | 18.56 | 18.69 | | 18.68 | |
| | GAPDH | 16.83 | 16.94 | 100 | 16.81 | 102 |
| | | 16.76 | 16.85 | | 16.77 | |
| Marker of cell | KRT19 | 27.88 | 27.85 | 109 | 25.77 | 446 |
| regeneration/ | | 27.93 | 27.91 | | 25.77 | |
| differentiation | | | | | | |
| Desquamation | KLK7 | 21.10 | 21.49 | 80 | 20.93 | 112 |
| | | 21.06 | 21.53 | | 20.96 | |
| Hydration | FLG | 26.46 | 26.85 | 82 | 25.64 | 185 |
| | | 26.48 | 26.86 | | 25.57 | |
| | AQP3 | 22.57 | 22.56 | 102 | 22.75 | 91 |
| | | 22.52 | 22.67 | | 22.65 | |
| Innate immunity | S100A7 | 27.26 | 28.01 | 66 | 27.82 | 76 |
| | | 27.60 | 28.22 | | 27.85 | |
| Response to oxidative | HMOX1 | 26.99 | 26.91 | 116 | 25.54 | 270 |
| and cellular stress | | 26.98 | 26.84 | | 25.61 | |
| Barrier function | TGM1 | 23.42 | 23.75 | 83 | 22.96 | 136 |
| | | 23.35 | 23.74 | | 22.96 | |

Under the experimental conditions of this study, the compounds beech bud oil macerate (tested at 0.1%), beech bud aqueous extract (tested at 0.3%) and white birch sap (tested at 1%) did not overall modulate the expression of the genes analyzed.

The white birch sap compound tested in combination with beech bud oily macerate induced an increase in the expression of genes involved in cell proliferation (KRT19), barrier/hydration function (FLG) and response to oxidative and cellular stress (HMOX1). Treatment of keratinocytes with the white birch sap compound tested in combination with aqueous beech bud extract significantly increased the expression of markers KRT19 (involved in cell proliferation), KLK7 (involved in desquamation), FLG (involved in barrier function/hydration), HMOX1 (involved in oxidative and cellular stress response) and TGM1 (involved in barrier function).

Under the experimental conditions of this study, the compound white birch sap (tested at 1%), tested in combination with beech bud aqueous extract (tested at 0.3%) or beech bud oily macerate (tested at 0.1%) showed a significant synergistic effect on modulating keratinocyte gene expression.

### CONCLUSION

The compound white birch sap (tested at 1%), tested in combination with the compound beech buds oily macerate (tested at 0.1%) induced a synergistic effect on the modulation of keratinocyte gene expression in epidermal regeneration, barrier/hydration function and protection against oxidative stress, thus against skin ageing.

The compound white birch sap (tested at 1%), tested in combination with the compound beech buds aqueous extract (tested at 0.3%), induced a significant synergistic effect on the modulation of keratinocyte gene expression involved in epidermal regeneration, desquamation, barrier/hydration function and protection against oxidative stress and therefore skin ageing.

### Example 2: Formulation of a Cosmetic Composition of the Invention (Day Fluid)

TABLE 10

| INCI name | % material - finished product |
|---|---|
| water | Between 50% and 70%. |
| glycerin | Between 7% and 10%. |
| oils | Between 7% and 12 |
| other extracts | Between 0.4% and 1%. |
| fragrance | Between 1% and 2% |
| alcohol | Between 0.05% and 0.08%. |
| fagus sylvatica bud oil extract | Between 0.2% and 5%. |
| fagus sylvatica aqueous bud extract | Between 0.2% and 5%. |
| betula alba sap | Between 0.5% and 6%. |
| Other excipients | Between 25% and 33 |
| TOTAL | 100% |

### LIST OF REFERENCES

1. Farid Chemat: Eco-extraction of plants, Editions Dunod, 2011

1. Cosmetic composition comprising at least one birch sap extract, and at least one extract chosen from an aqueous extract of beech bud and an oily extract of beech bud, and at least one cosmetically acceptable vehicle.

2. Cosmetic composition according to claim 1 comprising at least one birch sap extract and one beech bud aqueous extract, and optionally one beech bud oil extract.

3. Composition according to claim 1, said composition comprising at least one birch sap extract, one aqueous beech bud extract and one oily beech bud extract.

4. Composition according to claim 1, wherein said birch sap extract is a glycerin extract.

5. Composition according to claim 1, comprising from 0.2 to 5% by weight of said aqueous extract of beech bud relative to the total weight of the composition.

6. Composition according to claim 1, comprising from 0.2 to 5% by weight of said beech bud oil extract relative to the total weight of the composition.

7. Composition according to claim 1, comprising from 0.5 to 6% by weight of said birch sap glycerin extract relative to the total weight of the composition.

8. A composition according to claim 1, said composition being in a form selected from an ointment, cream, oil, milk, ointment, powder, soaked pad, solution, gel, serum, balm, butter, lotion, suspension, soap and emulsion.

9. A device in a form chosen from a jar, a pump bottle, a wipe, a mask, a transdermal device, a patch, a spray, a capsule or a softgel, said device comprising a cosmetic composition according to claim 1.

10. Method of non-therapeutic cosmetic care, comprising the application to the skin and/or its appendages and/or the mucous membranes of a cosmetic composition according to claim 1.

11. Method according to claim 10, wherein said application improves hydration, nutrition, oxygenation and stimulation of the protective barrier function of the skin and/or its appendages and/or mucous membranes.
